(19) United States
(12) Patent Application Publication
(10) Pub. No.: US 2012/0330420 A1
(43) Pub. Date: Dec. 27, 2012

(54) SPINAL FUSION IMPLANTS

(75) Inventors: Darrel S. Brodke, Salt Lake City, UT (US); Bret M. Berry, Tallahasee, FL (US); Ashok C. Khandkar, Salt Lake City, UT (US); Ramaswamy Lakshminarayanan, Salt Lake City, UT (US); Mahendra S. Rao, Timonium, MD (US)

(73) Assignee: Amedica Corporation, Salt Lake City, UT (US)

(21) Appl. No.: 13/468,278

(22) Filed: May 10, 2012

Related U.S. Application Data
(63) Continuation-in-part of application No. 12/800,219, filed on May 10, 2010, now abandoned.
Application No. 12/800,219 is a division of application No. 10/941,620, filed on Sep. 14, 2004, now abandoned.
Application No. 10/941,620 is a continuation-in-part of application No. 10/137,108, filed on Apr. 30, 2002, now Pat. No. 6,790,233.
(60) Provisional application No. 60/287,824, filed on May 1, 2001.

(51) Int. Cl. A61F 2/44

(52) U.S. Cl. 623/17.16

(57) ABSTRACT

An improved implant is provided for human implantation, such as a spinal implant for implantation into the intervertebral space between two adjacent vertebrae. Some embodiments include a substrate of high strength biocompatible material, such as doped silicon nitride ceramic for example. In some embodiments, the substrate may also include one or more regions of a controlled porosity analogous to natural bone. In other embodiments, the substrate may comprise the entire implant.

### RELATED APPLICATIONS

This is a continuation-in-part of copending U.S. patent application Ser. No. 12/800,219, filed May 10, 2010, which is a divisional of U.S. patent application Ser. No. 10/941,620, filed Sep. 14, 2004, which is a continuation-in-part of U.S. patent application Ser. No. 10/137,108, filed Apr. 30, 2002, now issued as U.S. Pat. No. 6,790,233, which claims the benefit of U.S. Provisional Patent Application No. 60/287,824, filed May 1, 2001. Each of the foregoing applications is hereby incorporated by reference.

### SUMMARY

In some embodiments disclosed herein, an improved implant, such as a spinal implant, is provided for human implantation into the space between a pair of adjacent vertebrae. Such spinal implants are typically installed following removal of disc material between endplates of the adjacent vertebrae to maintain the adjacent vertebrae in a predetermined and substantially fixed spaced relation while promoting interbody bone ingrowth and fusion. In this regard, some embodiments are designed for use in addressing clinical problems indicated by medical treatment of degenerative disc disease, discogenic lower back pain, and spondylolisthesis.

Some embodiments comprise a substrate block formed from a bio-compatible material composition having a relatively high bio-mechanical strength and load bearing capacity. This substrate may be porous, open-celled, or dense solid. A preferred composition of the high strength substrate block comprises a silicon nitride ceramic material. In some embodiments, the substrate may comprise the entire spinal implant. In other words, the substrate need not necessarily include another layer, material, or coating. In some such embodiments, the substrate (and therefore the entire implant) may comprise a solid, non-porous ceramic material, such as a silicon nitride ceramic material. These embodiments may comprise a solid block of non-porous ceramic or, alternatively, may comprise a non-porous ceramic having one or more openings extending through the top, bottom, and/or side surfaces to facilitate bone ingrowth.

In other embodiments, the substrate block may be porous. For example, some embodiments may have a porosity of about 10% to about 80% by volume with open pores distributed throughout and a pore size range of from about 5 to about 500 microns. When the substrate is porous, the porosity of the substrate block may be gradated from a first relatively low porosity region emulating or mimicking the porosity of cortical bone to a second relatively higher porosity region emulating or mimicking the porosity of cancellous bone. In other embodiments, as discussed above, the substrate block may comprise a dense solid comprised of a ceramic, metal or polymer material, with or without other materials, layers, or coatings. In some embodiments, this dense solid substrate may be attached to a second highly porous region emulating or mimicking the porosity of cancellous bone. In some embodiments, one or more of these porous regions would be formed around the substrate. However, in other embodiments, as discussed above, the substrate itself may comprise the entire implant.

In methods wherein a dense, solid material is used as the substrate block, the block may be externally coated with a bio-active surface coating material selected for relatively high osteoconductive and osteoinductive properties, such as a hydroxyapatite or a calcium phosphate material. The porous portion may be internally and externally coated with a bio-active surface coating material selected for relatively high osteoconductive and osteoinductive properties, such as a hydroxyapatite or a calcium phosphate material. The porous region or a portion of the porous region, however, may be in and of itself a bio-active material selected for relatively high osteoconductive and osteoinductive properties, such as a hydroxyapatite or a calcium phosphate material.

The implant can be made in a variety of shapes and sizes to suit different specific implantation requirements. Preferred shapes include a generally rectangular block with a tapered or lordotic cross section to suit the required curvature of the inter-vertebral space, in the case of a spinal fusion device. The exterior superior and inferior surfaces of the rectangular body may include ridges, teeth, spikes or other engagement features for facilitated engagement with the adjacent vertebrae. Alternative preferred shapes include a generally oblong, rectangular block which may also include serrations or the like on one or more exterior faces thereof, and/or may have a tapered or lordotic cross section for improved fit into the inter-vertebral space. A further preferred shape may include a crescent or cashew shape block which may also include serrations or the like on one or more exterior faces thereof, and/or may have a tapered or lordotic cross section for improved fit into the inter-vertebral space. The implant may desirably include notches for releasable engagement with a suitable insertion tool. In addition, the bone graft may also include one or more laterally open recesses or bores for receiving and supporting osteoconductive bone graft material, such as allograft (donor) or autograft (patient) material. Other openings may be provided that extend through the top and/or bottom surfaces of the implant.

Further alternative implant configurations may include a dense substrate region substantially emulating cortical bone, to define a high strength load bearing zone or strut for absorbing impaction and insertion load, in combination with one or more relatively high porosity second regions substantially emulating cancellous bone for contacting adjacent patient bone for enhanced bone ingrowth and fusion.

Some embodiments may exhibit a relatively high mechanical strength for load bearing support, for example, between adjacent vertebrae in the case of a spinal fusion implant, while additionally and desirably providing high osteoconductive and osteoinductive properties to achieve enhanced bone ingrowth and interbody fusion. In some embodiments, these desirable characteristics are achieved in a structure which is substantially radiolucent so that the implant does not interfere with post-operative radiographic monitoring of the fusion process.

Some embodiments may additionally carry one or more therapeutic agents for achieving further enhanced bone fusion and ingrowth. Such therapeutic agents may include natural or synthetic therapeutic agents such as bone morphogenic proteins (BMPs), growth factors, bone marrow aspirate, stem cells, progenitor cells, antibiotics, or other osteoconductive, osteoinductive, osteogenic, or any other fusion enhancing material or beneficial therapeutic agent.

Other features and advantages of various embodiments of the invention will become more apparent from the following detailed description, taken in conjunction with the accompanying drawings which illustrate, by way of example, some of the principles of preferred embodiments of the invention.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a perspective view depicting the spinal fusion cage in the inter-vertebral space;

FIG. 2 is a perspective view showing one preferred embodiment of the spinal fusion cage;

FIG. 3 is a perspective view showing the load bearing portion of the device of FIG. 2 with anterior and posterior load bearing walls connected by a strut, relieved in the superior and inferior aspects;

FIG. 4 is a perspective view depicting one alternative preferred and generally rectangular bone graft such as a spinal fusion cage;

FIG. 5 is a perspective view depicting the load bearing portion of the device of FIG. 4 with anterior and posterior load bearing walls connected by a strut, relieved in the superior and inferior aspects;

FIG. 6 is a perspective view showing still another alternative preferred form of the invention, comprising a generally oblong, rectangular bone graft such as a spinal fusion cage;

FIG. 7 is a perspective view depicting the load bearing portion of the device of FIG. 6 with anterior and posterior load bearing walls connected by a strut, relieved in the superior and inferior aspects;

FIG. 8 is an axial view of still another alternative form of the invention, taken generally on the load bearing axis of the spine, comprising a generally crescent shaped device conforming to the natural vertebral body shape;

FIG. 9 is a perspective view of the device of FIG. 8, showing a porous posterior margin;

FIG. 10 is a perspective view of the load bearing portion of the device of FIG. 8, showing anterior and lateral load bearing walls connected by a central strut, relieved in the superior and inferior aspects;

FIG. 11 is an axial view of a further preferred alternative embodiment of the invention, comprising a generally rectangular shape with macro-pores;

FIG. 12 is a perspective view of the device of FIG. 11 showing the interconnection of the macro-pores; and

FIG. 13 is a sectional view of the device of FIG. 11 taken generally along the mid-transverse plane 6-6 of FIG. 11 of the device.

### DETAILED DESCRIPTION OF THE PREFERRED EMBODIMENTS

As shown in the exemplary drawings, a radiolucent bone graft such as a spinal fusion implant referred to generally in FIGS. 1-3 by the reference numeral 10 is provided for seated implantation between a pair of adjacent patient bones such as spinal vertebrae 12 (FIG. 1) to maintain the vertebrae in spaced relation while promoting interbody bone ingrowth and fusion. In general, the spinal fusion implant 10 comprises a bio-compatible substrate. In some embodiments, the substrate makes up the entire implant. In other embodiments, the substrate may instead comprise an element upon which other materials, layers, coatings, etc. may be placed/formed. In some embodiments, the substrate may have a porous construction to define an open lattice conducive to interbody bone ingrowth and fusion, while providing a strong mechanical load bearing structure analogous to the load bearing properties of cortical and cancellous bone. This open-celled substrate may be coated internally and externally with a bio-active surface coating selected for relatively strong osteoconductive and osteoinductive properties, whereby the coated substrate provides a scaffold conducive to cell attachment and proliferation to promote interbody bone ingrowth and fusion attachment. The substrate may also carry one or more selected therapeutic agents suitable for bone repair, augmentation and other orthopedic uses.

FIGS. 1-3 illustrate an example of an improved spinal fusion implant 10 in accordance with one embodiment, in the shape of a generally rectangular body having ridges formed on the exposed top and bottom ends or faces 14. The lateral, anterior, and posterior walls of the body have notches 18 for the releasable engagement with an insertion tool.

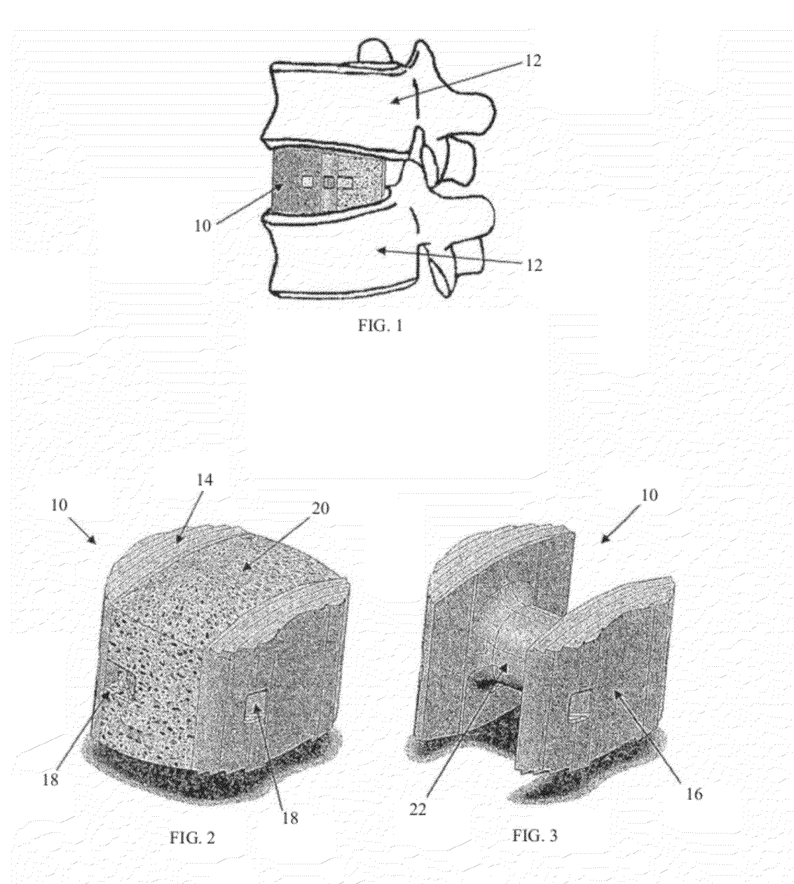

In some embodiments, the substrate composition comprises a relatively high strength block 16 (FIG. 3). In accordance with some embodiments, this substrate block 16 comprises a relatively dense silicon nitride composition having a controlled porosity (in some cases no porosity or substantially no porosity) and having a suitable size and shape for seated implantation, such as into the inter-vertebral space in the case of the spinal fusion implant 10. In some embodiments, the remainder of the substrate is comprised of a relatively porous silicon nitride 20 (FIG. 2) having an open-celled controlled porosity. One preferred silicon nitride ceramic material comprises a doped silicon nitride of the type disclosed in U.S. Pat. No. 6,881,229, which is incorporated by reference herein.

Moreover, in some embodiments, the pores are arranged with a variable porosity gradient to define a first region of relatively low or reduced porosity (less than about 5%) substantially mimicking cortical bone structure and a second region of relatively large or increased porosity (ranging from about 30% to about 80%) substantially mimicking cancellous bone structure. In one configuration, the outer or external surfaces of the reticulated substrate block comprise the first or low porosity region for improved load bearing capacity, while the interior surfaces of the substrate block comprises the second or high porosity region mimicking cancellous bone for enhanced bone ingrowth and fusion.

This high strength substrate block may be surface-coated internally and/or externally with a bio-active organic or inorganic surface coating material selected for relatively strong osteoconductive and osteoinductive properties to provide a nutrient rich environment for cellular activity to promote interbody bone ingrowth and fusion attachment. Preferred surface coating materials comprise a resorbable material, such as hydroxyapatite or a calcium phosphate ceramic. Alternative glassy (amorphous) materials having a relatively rich calcium and phosphate composition may also be used, particularly wherein such materials incorporate calcium and phosphate in a ratio similar to natural bone or hydroxyapatite. Such glassy compositions may comprise a partially or fully amorphous osteoinductive material comprising a composite of a glass and osteoinductive calcium compound, with a composition varying from about 100% glass to 100% osteoinductive calcium compound. The surface coating may also comprise autologous bone marrow aspirates.

The resultant spinal implant 10 may thus, in some embodiments, comprise the substrate block formed from the high strength material having bio-mimetic properties and which is nonresorbable, or slowly or infinitely slowly resorbable when implanted into the patient, in combination with the bio-active surface coating which is comparatively rapidly resorbable to promote rapid and vigorous bone ingrowth activity.

The substrate block may also advantageously be coated or impregnated with one or more selected therapeutic agents, for example, such as autologous, synthetic or stem cell derived growth factors or proteins and growth factors such as bone morphogenic protein (BMP) or a precursor thereto, which further promotes healing, fusion and growth. Alternative therapeutic agents may also include an antibiotic, or natural therapeutic agents such as bone marrow aspirates, and growth factors or progenitor cells such as mesenchymal stem cells, hematopoietic cells, or embryonic stem cells, either alone or as a combination of different beneficial agents.

The resultant illustrative spinal implant 10 may exhibit relatively high bio-mechanical strength similar to the load bearing characteristics of natural bone. In addition, the spinal implant 10 may exhibit relatively strong osteoconductive and osteoinductive characteristics attributable primarily to the surface coating, again similar to natural bone. The spinal implant 10 may also be substantially radiolucent, so that the implant does not interfere with post-operative radiological analysis of interbody bone ingrowth and fusion.

The relatively dense, high strength block 16 may be formed in a manner and with exposed faces or ends 14 with which to withstand the axial loading of the spine. In the embodiment as shown, the anterior and posterior walls of the device are formed as part of this high strength portion, each with exposed upper and lower ends or faces 14. This may be done to allow the high strength region to interface with the cortical ring of the adjacent vertebral body 12. Additionally, a strut 22 of the high strength material may extend between the anterior and posterior walls, which beneficially provides a load bearing structure capable of withstanding impaction and insertion loading in the anterior-posterior direction. Consequently, the relatively porous portion may be formed in between the dense anterior-posterior walls and around the central strut. The porous portion may thereby form the remainder of the device, including a large region of the superior, inferior, and lateral aspects. The porous portion, being less dense in nature than the high strength regions of the device, is increasingly radiolucent, thus allowing for assessment of bone growth and bony attachment to the adjacent vertebral body.

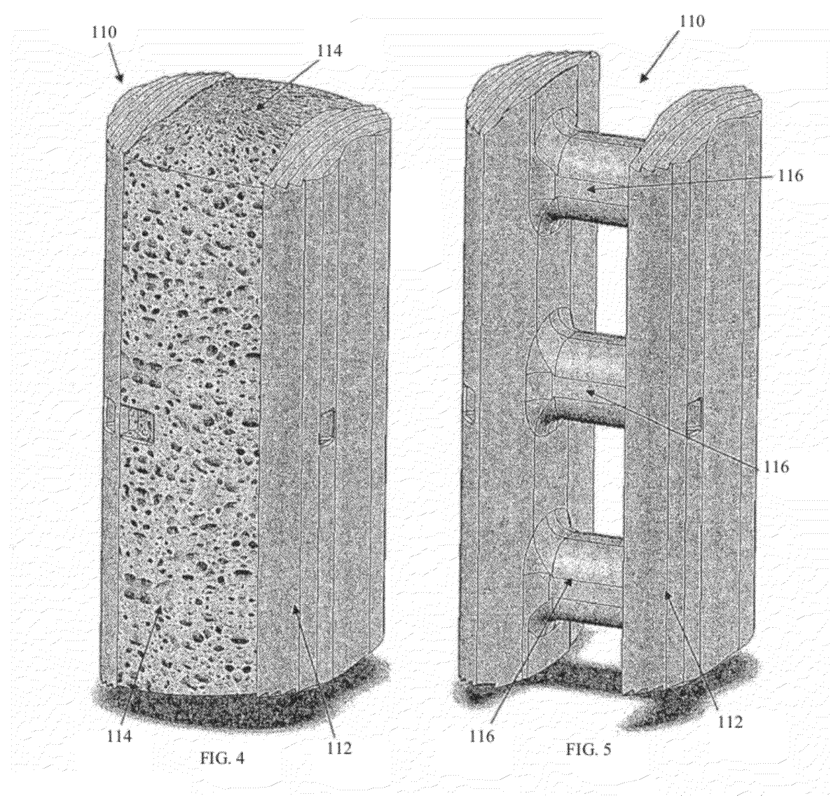

FIGS. 4-10 illustrate alternative configurations for improved bone grafts such as spinal fusion cages constructed in accordance with various embodiments and/or principles of the present invention, it being recognized and understood that the implant can be constructed in a wide range of different geometric sizes and shapes. FIG. 4 shows a spinal implant 110 having a generally rectangular shape similar to the implant 10 shown and described in FIGS. 1-3, but the form is elongated, as for use in replacing an entire vertebral body. As shown, the spinal implant 110 (FIG. 5) may have a relatively dense structure defined by a high strength substrate block 112 (as previously described) coated with the bio-active surface coating material, but wherein the relatively dense interior structure may be defined by multiple struts 116 with high strength for withstanding impaction and insertion loading in an anterior-posterior direction between anterior and posterior walls with exposed upper and lower ends or faces. The multiple struts 116 additionally may create interior openings which provide for lateral fluid transmission and optimize bone growth laterally through the center of the implant. FIG. 5 shows multiple dense struts, thereby demonstrating that the porous region is able to make contact with the adjacent superior and inferior vertebrae. The porous region 114 may be more radiolucent than the surrounding dense portion and therefore provides enhanced visualization for analysis of bone growth and subsequent fusion with the adjacent vertebrae. Each of the embodiments depicted in FIGS. 1-13 may have a height dimension and may be tapered or lordotic in shape for enhanced anatomical fit, for example, into the inter-vertebral space or the like.

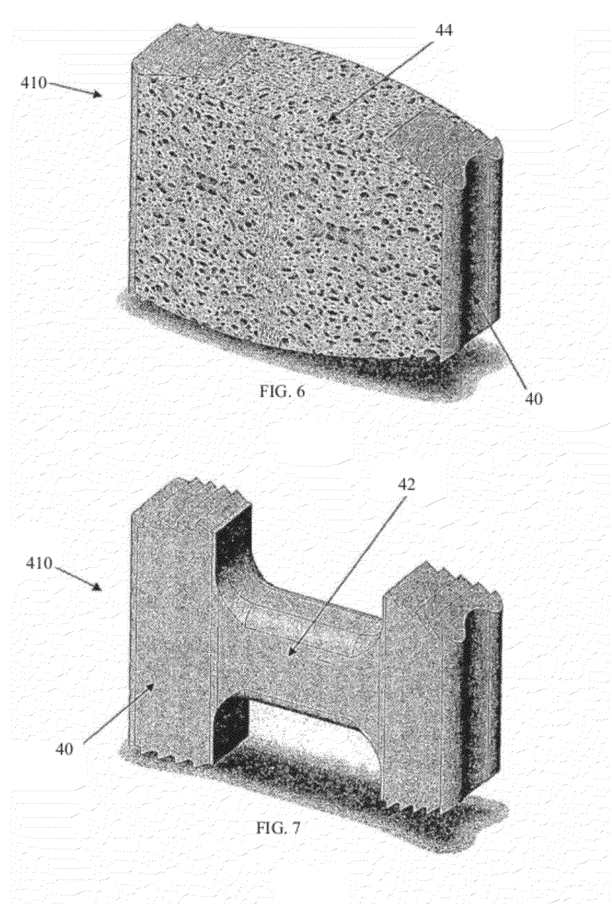

FIGS. 6-7 depict still another alternative embodiment of a spinal implant 410 having a generally oblong, rectangular geometry having a high strength, dense or first region 40, as well as a relatively porous or second region 44 for bone in-growth. This geometry may be useful for surgical approaches in which it is necessary to place two implants next to each other in the intervertebral space. More particularly, FIGS. 6-7 show a generally oblong, rectangular spinal implant 410 having a tapered height dimension in the anterior-posterior direction.

The substrate block may be formed with the first region 40 of relatively low porosity substantially mimicking cortical bone to extend across the anterior and posterior faces and further to include at least one interconnecting load bearing strut 42 shown in the illustrative drawings to extend centrally in an anterior-posterior direction within the body of the substrate block. Of course, in other embodiments, a plurality of such struts may be provided. The remainder of the substrate block may comprise a second portion 44 of relatively high porosity substantially mimicking cancellous bone.

The harder first region 40 including the central strut 42 beneficially provides a hard and strong load bearing structure analogous to that shown and described with respect to FIGS. 1-5, and capable of withstanding impaction and insertion forces in the anterior-posterior direction without damage to the implant, while the softer second region 44 may provide an exposed and large surface area for substantially optimized interknitting ingrowth and fusion with adjacent patient bone. In a spinal implant application, the medial-lateral faces of the implant may be defined by the softer, more porous second region 44, since these regions are often exposed to traditional medial-lateral X-ray imaging for post-operative radiological analysis of the implant/bone interface. Persons skilled in the art will recognize and appreciate that alternative configurations for the load bearing strut or struts 42 may be used, such as an X-shaped strut configuration extending in a cranial-caudial direction, in combination with or in lieu of the exterior faces of dense region 40 and/or the anterior-posterior central strut as shown.

Of course, in other embodiments, these struts may be removed altogether. In addition, in some embodiments, the spinal implant may be entirely, or substantially entirely, made up of a single material having a substantially constant density, hardness, and/or porosity. For example, in some embodiments, the entire spinal implant may comprise a dense, non-porous silicon nitride substrate without any additional layers, materials, or coatings that have a higher porosity. Of course, still other embodiments are contemplated in which the entire spinal implant may comprise a somewhat porous substrate with or without additional layers, materials, coatings, etc., having differing densities/porosities.

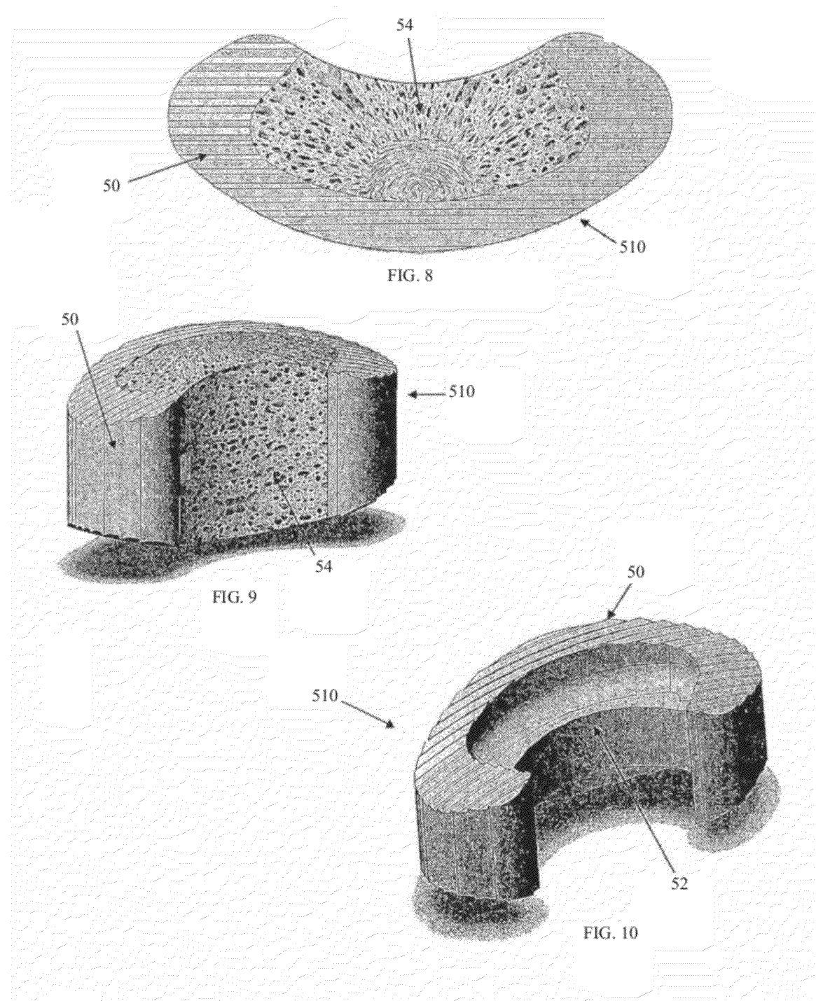

FIGS. 8-10 depict a further alternative embodiment of the invention, with a generally crescent-shaped geometry 510. The substrate block of the embodiment in these figures may be formed of a relatively dense, high strength region 50 substantially mimicking cortical bone extending along the anterior and lateral walls and including exposed upper and lower ends or faces. The dense portion 50 may provide a strong load bearing structure capable of withstanding axial loads in the spine. Also, the high-strength region 50 may be located along the anterior side of the substrate, thereby interfacing with the load bearing cortical bone of the adjacent vertebral body. An integral dense strut 52 extends between the dense lateral walls providing a load bearing structure for impaction and insertion forces exhibited in a lateral approach. The superior, inferior, and posterior portions of the substrate may be formed with a relatively porous material region 54 to provide for bone growth and increased radiolucency.

In other embodiments, the dense, high strength region 50 may make up the entire substrate/implant. For example, region 54 may instead comprise an extension of region 50 such that the entire crescent-shaped implant 510 may be made up of solid, non-porous, and relatively dense silicon nitride ceramic material.

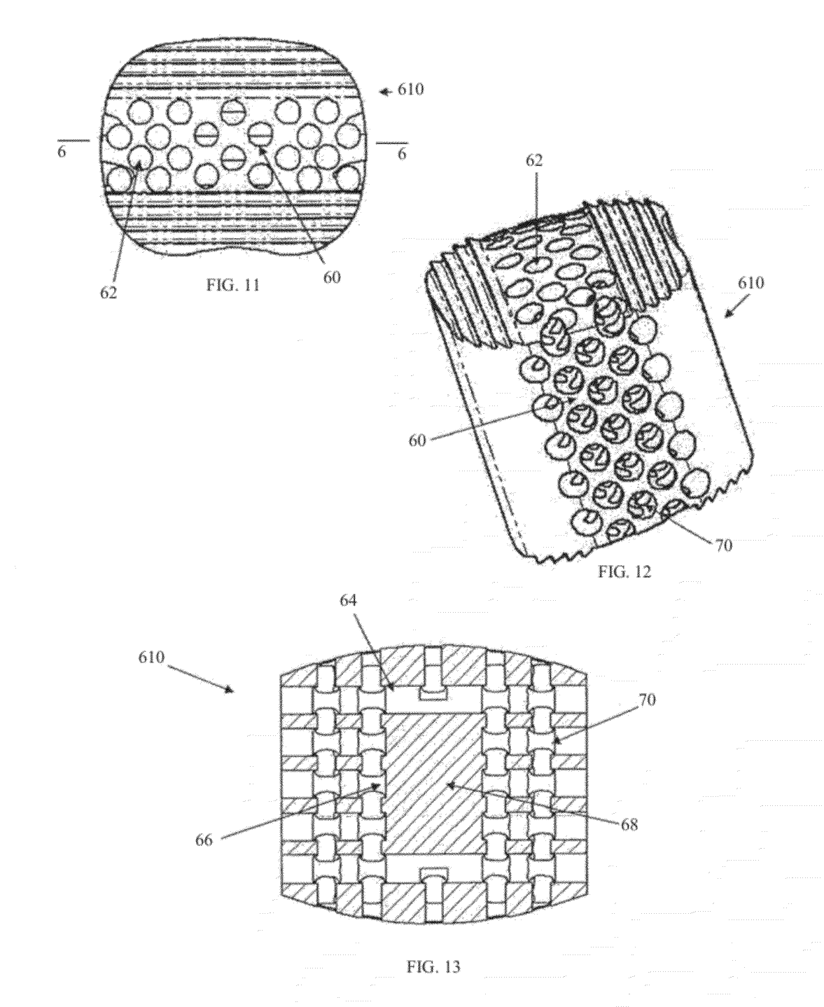

FIGS. 11-13 depict a still further alternative embodiment which is formed entirely of a relatively low porosity, high-strength substrate 610. The subsequent porous structure 60 may be created by drilling or boring a plurality of macro-pores 62 into the superior, inferior, and lateral faces of the device. This method allows the anterior and posterior walls to remain intact and thus be able to withstand the loading of the spinal column. The macro-pores may be oriented in both the axial direction of the spine, as well as between the lateral walls of the device, as shown in the figures. This may allow bone to grow in the direction of the spinal loading and also laterally through the substrate. The macropores may be positioned in such a manner as to allow for continuous interconnection 70, thereby creating a meshwork of pores for bony ingrowth into the device. Some or all of the macropores may extend from one face of the device to the opposite face 64, or towards the center of the device. Some or all of the macropores may further be extended to a certain depth, and terminated therein to form a blind macropore 66. The blind macropores 66 may create a portion in the center of the device which remains solid and therefore serves as a load bearing strut 68 extending from the anterior wall to the posterior wall to withstand impaction and insertion loads in the anterior-posterior direction. This macropore method can also be utilized with geometries similar to those depicted in FIGS. 6-10, such as the oblong rectangular implant 410 and the crescent implant 510.

In all of the embodiments of FIGS. 1-13, the substrate block may comprise a high strength porous or non-porous ceramic as previously described, such as a silicon nitride or doped silicon nitride ceramic. The substrate block may also be coated with a bio-active surface coating material, again as previously described, to enhance bone ingrowth and fusion. The substrate block may also include one or more therapeutic agents. Persons skilled in the art will recognize and appreciate that, in embodiments providing separate regions having differing porosities, the relatively low and high porosity regions 16 and 20 shown in FIGS. 2-3 may also be integrally joined by a suitable, albeit relatively narrow, gradient region wherein the porosity transitions therebetween.

The spinal implants disclosed herein may thus comprise an open-celled substrate block structure, or a dense, non-porous substrate block structure, which may be coated with a bio-active surface coating, and may be configured to have the strength required for the weight bearing capacity required of a fusion device. The capability of being infused with the appropriate biologic coating agent imparts desirable osteoconductive and osteoinductive properties to the device for enhanced interbody bone ingrowth and fusion, without detracting from essential load bearing characteristics. The radiolucent characteristics of some embodiments of the device beneficially accommodate post-operative radiological examination to monitor the bone ingrowth and fusion progress, substantially without radio-shadowing attributable to the spinal implant. The external serrations or threads formed on the spinal implant may have a variable depth to enable the base of the device to contact the cortical bone for optimal weight bearing capacity. In addition to these benefits, some embodiments may be easy to manufacture in a cost-competitive manner. Various embodiments may therefore provide a substantial improvement in addressing clinical problems indicated for medical treatment of degenerative disc disease, discogenic low back pain, and spondylolisthesis.

It will be understood by those having skill in the art that changes may be made to the details of the above-described embodiments without departing from the underlying principles presented herein. For example, any suitable combination of various embodiments, or the features thereof, is contemplated.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment. It will be apparent to those having skill in the art that changes may be made to the details of the above-described embodiments without departing from the underlying principles set forth herein.

A variety of further modifications and improvements in and to the spinal implants disclosed herein will be apparent to those persons skilled in the art. In this regard, it will be recognized and understood that the spinal implants can be formed in the size and shape of a small pellet for suitable packing of multiple implants into a bone regeneration/ingrowth site, or any of a wide variety of alternative shapes and sizes, as desired by a surgeon for a particular use or application. Accordingly, no limitation on the invention is intended by way of the foregoing description and accompanying drawings, except as set forth in the appended claims.

1. A spinal implant, comprising:
a block comprising a silicon nitride ceramic material, wherein at least a portion of the silicon nitride ceramic material has a flexural strength greater than about 700 Mega-Pascal (MPa), wherein the silicon nitride ceramic material has a toughness greater than about 7 Mega-Pascal root meter (MPam^{0.5}), and wherein the block comprises:
a top surface configured to support a first adjacent vertebral body;
a bottom surface configured to support a second adjacent vertebral body; and
one or more sidewalls extending between the top and bottom surfaces.

2. The spinal implant of claim 1, wherein the silicon nitride material is substantially non-porous.

3. The spinal implant of claim 1, wherein at least a portion of the silicon nitride material has a porosity substantially mimicking that of natural cortical bone.

4. The spinal implant of claim 1, wherein the top surface comprises an opening extending therethrough.

5. The spinal implant of claim 4, wherein the bottom surface comprises an opening extending therethrough.

6. The spinal implant of claim 5, wherein the top surface opening connects with the bottom surface opening such that at least one opening extends through both the top and bottom surfaces of the block.

7. The spinal implant of claim 1, wherein the block comprises a porous region.

8. The spinal implant of claim 7, wherein the porous region comprises, at least in part, a porosity substantially mimicking that of natural cancellous bone.

9. The spinal implant of claim 8, wherein the porosity of the implant is gradated from the porous region to a less porous region of the implant.

10. The spinal implant of claim 9, wherein the porosity of the implant is gradated from a portion of the implant having a porosity substantially mimicking that of cortical bone to the porous region.

11. The spinal implant of claim 10, wherein the porous region is positioned on an exterior surface of the implant.

12. The spinal implant of claim 1, wherein the block comprises a doped silicon nitride ceramic material.

13. The spinal implant of claim 12, wherein the doped silicon nitride ceramic material comprises dopants selected from the group consisting of yttrium oxide, magnesium oxide, strontium oxide, aluminum oxide, and combinations thereof.

14. The spinal implant of claim 13, wherein the doped silicon nitride ceramic material comprises Silicon Nitride doped with about 6% yttrium oxide by weight and about 4% aluminum oxide by weight.

15. The spinal implant of claim 1, wherein the block comprises a substantially crescent shape.

16. The spinal implant of claim 1, wherein the top and bottom surfaces comprise a plurality of engagement features.

17. A spinal implant, comprising:
a substrate comprising a substantially non-porous doped silicon nitride ceramic material, wherein at least a portion of the doped silicon nitride ceramic material has a flexural strength greater than about 700 Mega-Pascal (MPa), wherein the doped silicon nitride ceramic material has a toughness greater than about 7 Mega-Pascal root meter (MPam^{0.5}), wherein the doped silicon nitride material comprises dopants selected from the group consisting of yttrium oxide, magnesium oxide, strontium oxide, aluminum oxide, and combinations thereof, and wherein the substrate comprises:
a top surface configured to support a first adjacent vertebral body, wherein the top surface comprises at least one opening extending therethrough;
a bottom surface configured to support a second adjacent vertebral body, wherein the bottom surface comprises at least one opening, and wherein at least one of the top surface openings connects with at least one of the bottom surface openings such that at least one opening extends all the way through the top surface and the bottom surface of the substrate; and
one or more sidewalls extending between the top and bottom surfaces.

18. The spinal implant of claim 17, wherein the doped silicon nitride ceramic material has a flexural strength greater than about 900 Mega-Pascal (MPa).

19. The spinal implant of claim 17, wherein the doped silicon nitride ceramic material has a toughness greater than about 9 Mega-Pascal root meter (MPam^{0.5}).

20. The spinal implant of claim 17, wherein the doped silicon nitride ceramic material comprises Silicon Nitride doped with yttrium oxide and aluminum oxide.

21. The spinal implant of claim 20, wherein the doped silicon nitride ceramic material comprises Silicon Nitride doped with about 6% yttrium oxide by weight and about 4% aluminum oxide by weight.
